(19) United States
(12) Patent Application Publication
(10) Pub. No.: US 2022/0007931 A1
(43) Pub. Date: Jan. 13, 2022

(54) Filter Test Kit and Filter Test Frame for Low Vision

(71) Applicant: Independence Low Vision, LLC, Phoenix, AZ (US)

(72) Inventor: Elizabeth Evans, Phoenix, AZ (US)

(21) Appl. No.: 17/365,409

(22) Filed: Jul. 1, 2021

Related U.S. Application Data
(60) Provisional application No. 63/049,235, filed on Jul. 8, 2020.

(51) Int. Cl. A61B 3/04; A61B 3/032

(52) U.S. Cl. CPC A61B 3/04; A61B 3/032

(57) ABSTRACT

A filter test kit for low vision is provided and may include a plurality of test frames. A test frame may include two sockets, a pair of optical lens tint components disposed in the sockets, a bridge connecting the sockets; and a tactile frame identifier. The pair of optical lens tint components for each frame is different from the pair of optical lens tint components of each other frame. Also, the tactile frame identifier for each frame is different from the tactile frame identifier for each other frame.

### CROSS REFERENCE TO RELATED APPLICATIONS

This application claims priority to U.S. provisional application No. 63/049,235, filed Jul. 8, 2020, the entire contents of which are incorporated herein by reference.

### BACKGROUND

Glare is reflected or uncontrolled light that shines directly into a person's eyes. Although very bright and intense, light produced by glare generally does not help a person see more clearly. Rather, glare can interfere with a person's visual comfort, physical safety and independent performance of activities of daily life. Glare can be particularly troublesome for older people, people who have had prior eye surgery, and those who have low vision eye conditions.

Certain people require colored light filters to reduce glare under different lighting conditions including indoors and/or outdoors. To determine whether a person needs colored light filtered lenses and which type of lens, an eye evaluation is typically performed by an eye doctor at their office or certified low vision technology (CLVT) therapist, or other professional using a set of filtered glasses or low vision flippers such as SCST Flippers sold by Bernell®.

### BRIEF SUMMARY

The following presents a simplified summary of various aspects described herein. This summary is not an extensive overview, and is not intended to identify key or critical elements or to delineate the scope of the claims. The following summary merely presents some concepts in a simplified form as an introductory prelude to the more detailed description provided below.

With the onset of COVID-19, patients have been restricted from coming to an eye doctor's office to be evaluated such that determining an appropriate lens filter for those with low vision has become challenging. For example, patients did not have access to a set of filtered glasses to determine the best lenses for their condition. In addition, doctors and CLVTs were not willing to send a set of filtered glasses to patients due to the expense of the set of glasses and the need to have the set of glasses returned and ultimately sanitized after each use. Also, it was cost prohibitive as well unreasonable to expect patients to purchase a set of filtered glasses, which would require a patient to, for example, purchase six pairs of filtered glasses. Even after people have been able to return to their doctor's office or meet in person with a CLVT, it has still been necessary to sanitize the testing equipment between each use during COVID-19. Sanitizing the testing equipment can be cumbersome and time-consuming, add significant expense, and limit the number of patients who could be tested due to the sanitization process. In any event, there are low vision patients who live in remote locations or who may not be able to easily travel to a see an eye doctor or CLVT in person to be evaluated to determine the most appropriate lenses.

To address these problems, a low-cost single use test kit was developed that is useable by a patient with low vision who may not even be able to recognize the filter colors. The kit can be used in a doctor's office, at a person's residence, or at another location remote from the doctor's office. That is, the development of the kit has allowed a single use test filter to be delivered by mail, courier or otherwise to a person (such as a patient or caregiver) at a location remote from a doctor's office or other testing location. The recipient of the kit can conduct a test with various glare control filters in different environments with different light settings. The kit can be for one time use and disposed of thereafter. Or, the kit may be saved and reused by the same patient during a future evaluation. The relative cost of the kit makes it reasonable to simply discard the kit after its use.

### BRIEF DESCRIPTION OF THE DRAWINGS

A more complete understanding of the present invention and the advantages thereof may be acquired by referring to the following description in consideration of the accompanying drawings, in which like reference numbers indicate like features, and wherein:

FIG. 1 illustrates a frame according to an illustrative implementation of the invention.

FIG. 2 illustrates an illustrative filter test kit for low vision according to an illustrative implementation of the invention.

FIG. 3 shows a tint shape color key example according to illustrative aspects of the invention.

FIG. 4 illustrates another example of a filter test kit for low vision according to an aspect of the invention.

FIG. 5 illustrates another example of a frame according to an aspect of the invention.

FIG. 6 illustrates an exploded view of a portion of the frame according to FIG. 5 with an optical component.

FIG. 7 illustrates a further example of a frame according to an aspect of the invention.

FIG. 8 illustrates an example of the frame according to FIG. 7 with the optical components.

### DETAILED DESCRIPTION

In the following description of the various embodiments, reference is made to the accompanying drawings, which form a part hereof, and in which is shown by way of illustration various embodiments in which aspects of the disclosure may be practiced. It is to be understood that other embodiments may be utilized and structural and functional modifications may be made without departing from the scope of the present disclosure. Aspects of the disclosure are capable of other embodiments and of being practiced or being carried out in various ways. Also, it is to be understood that the phraseology and terminology used herein are for the purpose of description and should not be regarded as limiting. Rather, the phrases and terms used herein are to be given their broadest interpretation and meaning. The use of “including” and “comprising” and variations thereof is meant to encompass the items listed thereafter and equivalents thereof as well as additional items and equivalents thereof.

A filter test kit for low vision according to illustrative aspects of the invention may be used in any lighting condition including, but not limited to, indoors, outdoors, in sunlight, in artificial light, and at nighttime. Aspects of the filter test kit can be used by a patient, CLVT or caregiver at the patient's residence or other location remote from a doctor's office.

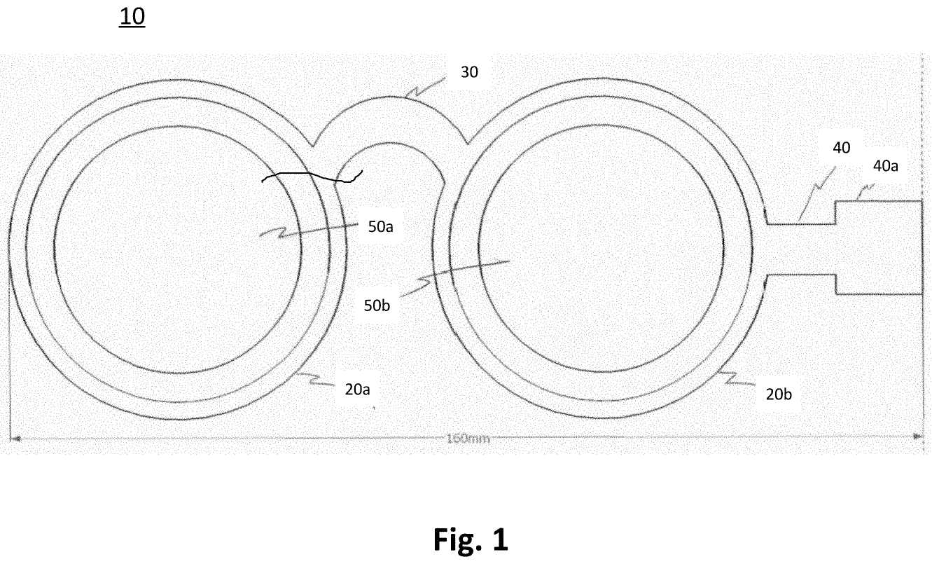

According to certain aspects, a filter test kit may include plural frames. Typically, a minimum of six different frames will be included in a kit, but it will be appreciated that more or less frames may be included as needed. Each frame 10 includes two sockets 20a, 20b, a bridge 30 connecting the two sockets 20a, 20b together, and a tab 40 protruding from a socket 20b. The sockets 20a, 20b are sized appropriately to be usable by a patient taking into consideration the general size of a human face and the size of a lens of glasses. For example, the diameter of the inner circumference of a socket may be approximately 43 mm and the radius of the socket (from the outer circumference) may be approximately 29.5 mm. In one example, the overall maximum length of the frame from the end of the tab 40 to the outside end of the socket 20a may be around 160 mm. The bridge 30 is sized appropriately to rest on a human nose when a user brings the frame 10 to an eyeglass wearing position to look through the sockets 20a, 20b. The sockets 20a, 20b are configured to hold optical components 50a, 50b. For low vision testing, the optical components 50a, 50b correspond to lens tints though FIG. 1 shows transparent optical components.

The tab 40, which includes a tactile frame identifier 40a, protrudes away from the socket 20b in FIG. 1 and allows a user or patient to hold the frame 10 up to their eyes. As illustrated, the tab 40 extends away from a side of one of the sockets in a direction away from the other of the sockets. An imaginary line intersecting the center of each of the sockets is parallel to the direction in which the tab 40 extends away from the socket 20b and passes through the tab 40 and the tactile frame identifier 40a of the tab 40. It will be appreciated that the tab 40 can extend away from the sockets 20a, 20b or bridge 30 in a direction away from the frame 10. For scenarios in which the patient is holding the frame 10 and taking into consideration facial and head structure of the patient, it has been found that the tab 40 extending away from the frame 10 as shown in FIG. 1 is intuitive and easy for patients with impaired vision.

The tactile frame identifier 40a allows a patient to identify the specific frame 10. That is, since many patients are not able to visually identify the specific optical components 50a, 50b of the frame 10 due to their low vision condition, a methodology is needed to allow a patient to identify the frame 10 including its pair of lens tint components. The lens tint components for each of the frames is a different color from the lens tint components of each other frame so that a patient can try each of the frames and it can be determined which lens tint components improves their vision under various indoor and outdoor conditions as well as natural light and artificial light conditions.

To allow the user to identify the specific pair of lens tint components in the frame 10, a unique tactile frame identifier 40a can be used such that each frame 10 in a kit has a different tactile frame identifier 40a. The tactile frame identifier 40a can take many forms including braille, surface granularity, providing a different number of protrusions or a well-known shape that is easily recognizable to a visually impaired person such as a geometric shape or other well-known shape like a heart. Alternatively, the frame identifier may further include a different number of small protrusions, indentations or concave and convex regions provided on each frame 10 in the kit. Any tactile form can be used although it has been determined in testing that different recognizable shapes work well. While the tactile frame identifier 40a is provided on the tab 40 of the frame 10 in FIG. 1, it will be appreciated that the tactile frame identifier may be provided on other surfaces of the frame 10 including the sockets 20a, 20b and the bridge 30. Though for ease of use for a visually impaired patient, it was practical to include the tactile frame identifier on the tab 40 so the patient can identify, at the time they are looking through the frame 10, the specific lens tint components they are looking through.

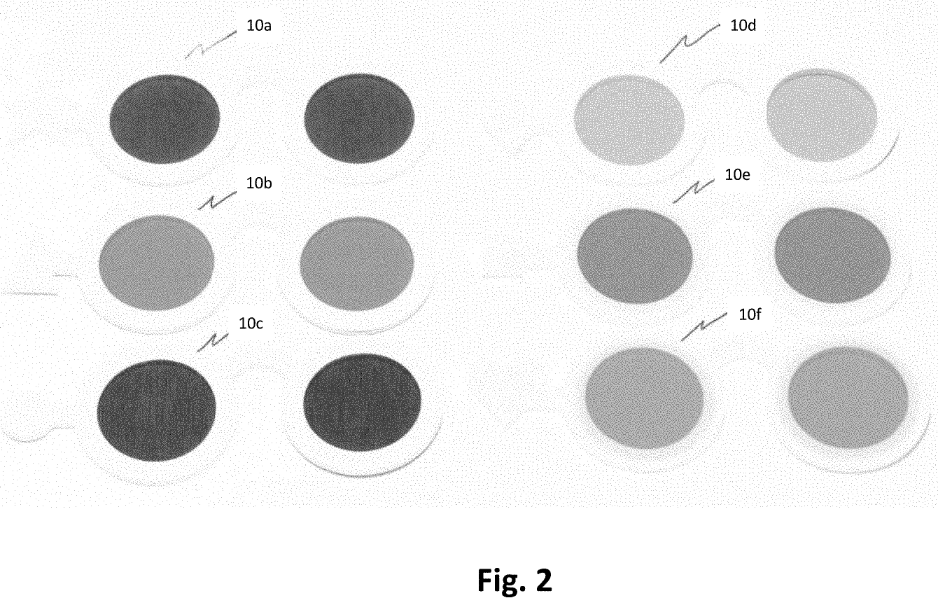

In one aspect, six frames 10a, 10b, 10c, 10d, 10e and 10f are part of the kit such as illustrated in FIG. 2. Each of the frames includes a tactile frame identifier (unlabeled). In this example, frames 10a, 10b, 10c, 10d, 10e and 10f respectively have a tactile frame identifier of a star shape, triangle shape, circle shape, diamond shape, square shape and heart shape. The lens tint components for each of the frames is a different color from the lens tint components of each other frame as shown.

In the FIG. 2 example, the color of the lens tint components for the frames 10a, 10b, 10c, 10d, 10e, and 10f respectively correspond to plum, amber, gray, yellow, green, and orange. In one example, for the lens tint components, Roscolux filters may be used. In this example, the Rosco brand Roscolux 50 Mauve Gel may be used as the plum lens tint component; the Rosco brand Roscolux 20 Medium Amber Gel may be used as the amber lens tint component; the Rosco brand Roscolux 98 Medium Grey Gel may be used as the lens tint component; the Rosco brand CalColor 4560-CalColor 60 Yellow Gel may be used as the yellow lens tint component; a combination (e.g., one on top of the other) of Rosco brand Roscolux 398 Neutral Grey Gel and Rosco brand Roscolux 12 Straw Gel may be used as the green lens tint component; and the Rosco brand Roscolux 3152 Urban Vapor Gel may be used as the orange lens tint component. It will be appreciated that other lens filters can be used as needed or instead of the above from Rosco or another lens filter manufacturer.

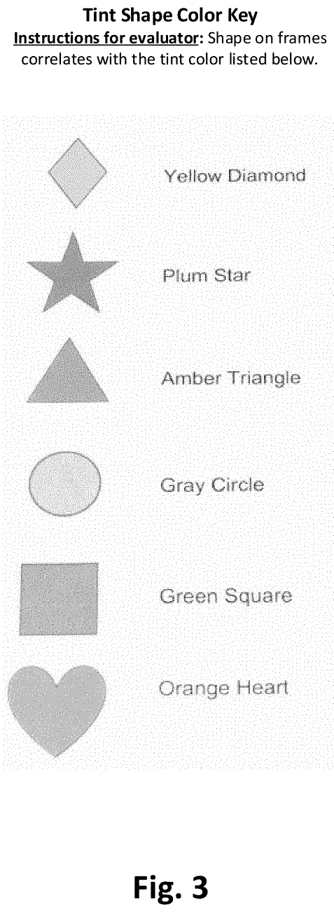

In one example of using the kit, the patient can try out each of the frames 10a, 10b, 10c, 10d, 10e, and 10f to determine if glare is reduced in a particular setting and environment, and the results may be recorded on a tint shape color key form (which may be in braille). An example tint shape color key is illustrated in FIG. 3. In this example, the tint shape color key identifies for each shape of the tactile frame identifier the corresponding lens tint component. The patient can identify the performance and the environment on the tint shape color key using one key or one key per environment. For maximum benefit, the glare filter test needs to be performed indoors/outdoors, in sunlight, in artificial light, at nighttime or any other lighting conditions the individual might encounter. This helps the evaluator determine what filter will help increase the individual's contrast sense which will also increase their visually acuity. Alternatively, the patient can share their assessment of each of the frames over the phone with a doctor, CLVT or other appropriate professional.

In another example of using the kit, a doctor, CLVT, or other professional may, over the phone, provide directions to the patient for performing the low vision test and instruct the patient to try the frame with the square tactile frame identifier or another specific shape so that the particular lens filter performance for the patient can be tracked. Eventually, after each of the frames are tested, the most suitable lens filter can be identified across all the conditions or for a specific condition, and an appropriate pair of glasses can be prescribed for the patient.

Each of the frames 10a, 10b, 10c, 10d, 10e, and 10f may be manufactured via 3D printing using, for example, PLA (polyactide), which is a thermoplastic polyester. PLA is a relatively inexpensive material. Other alternative materials that may be used for the frame include ABS (acrylonitrile butadiene styrene), PETG (polyethylene terephthalate (with a glycol modification), nylon as well as most other non-flexible plastics. Since the filter kits are for one-time use, it is important to use a cost-effective material and manufacturing process.

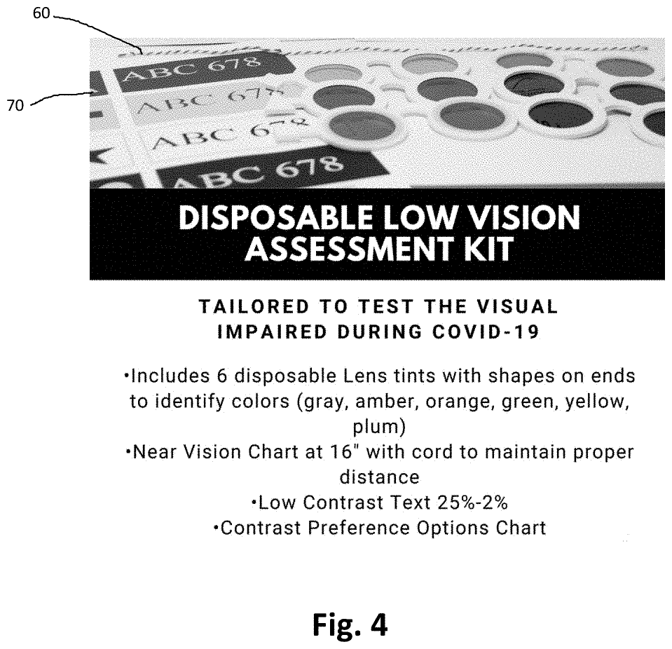

An illustrative filter test kit for low vision is shown in FIG. 4 and may include items in addition to the frames (not labeled). For example, the kit may include a distance indicator 60 such as an 8 inch or 16 inch (20 cm or 40 cm) long cord to help the patient or user maintain the proper distance from an eye chart to perform the test of each of the frames. For a patient with 20/400 vision, 16 inches (40 cm) is an appropriate distance. For patients with 20/800 vision or worse, the test should be performed at 8 inches (or 20 cm) from the eye chart. It will be appreciated that other items may be used as a distance indicator including string or other thread base materials. While a more rigid distance indicator may be used, a soft material indicator is much preferred since the a visually impaired patient could scratch, poke or otherwise injure themselves inadvertently when trying to make sure the proper test condition is in place. The kit may include an eye chart 70 such as a contrast preference chart with low contrast text (25%-2%). Alternatively, an eye chart may be obtained from other sources.

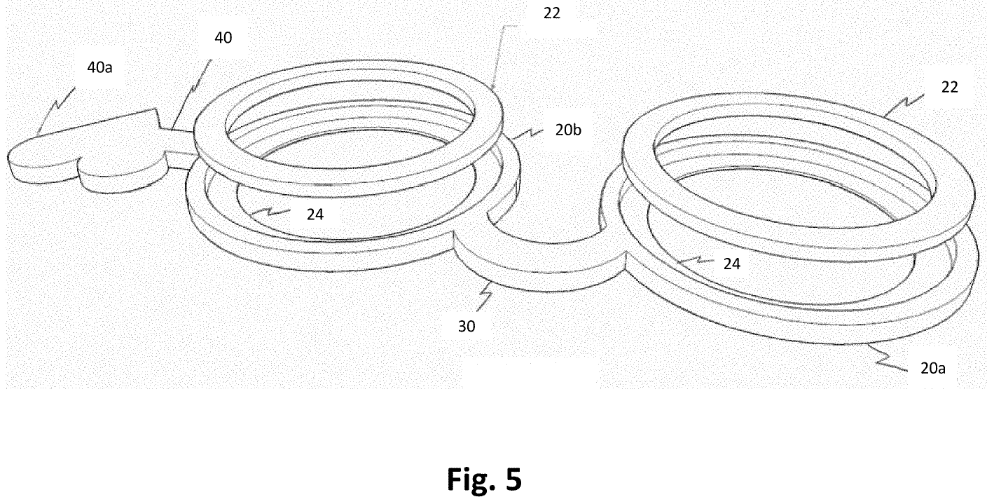

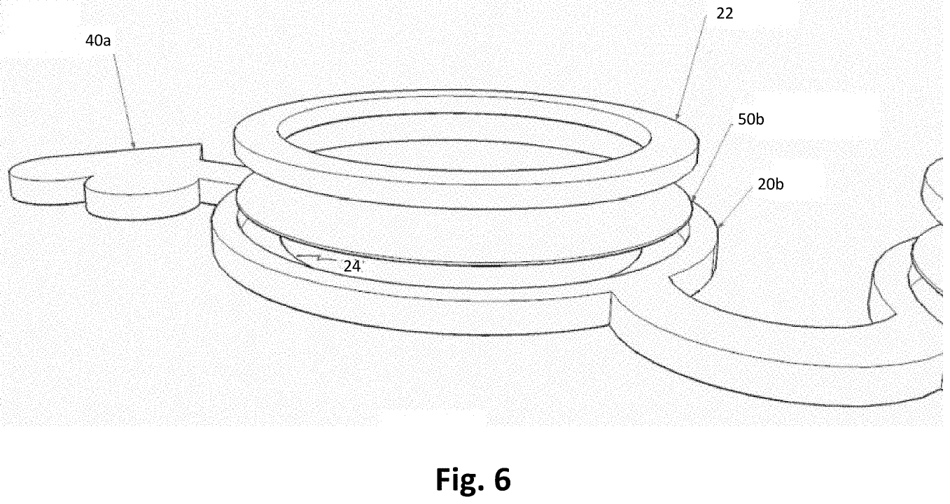

In an illustrative frame 10 as shown in FIG. 5, the sockets 20a, 20b may each include a ring insert 22 that press fits into the circular base 24 of the respective socket. During assembly of the frame 10, an optical component (e.g., 50b) is placed inside the circular base 24 of the socket 20b such that the ring insert 22 holds the optical component within the frame 10 when the ring insert 22 is press fit into the circular base 24 of the socket 20b. FIG. 6 illustrates an exploded view of a portion of the frame including one of the sockets with an optical component 50b between the ring insert 22 and the circular base 24. Typically, the kit would be shipped with the optical components fitted into the frame 10 since it would generally not be desirable for the patients to have to assemble the frame 10 prior to performing a low vision test. It will be appreciated however that, it is possible that the kit could be shipped unassembled or that the kit does not include the optical components. For example, if kits were shipped to a local facility for use with low income patients, the facility or individual performing the testing may provide the filters to be inserted into the various frames.

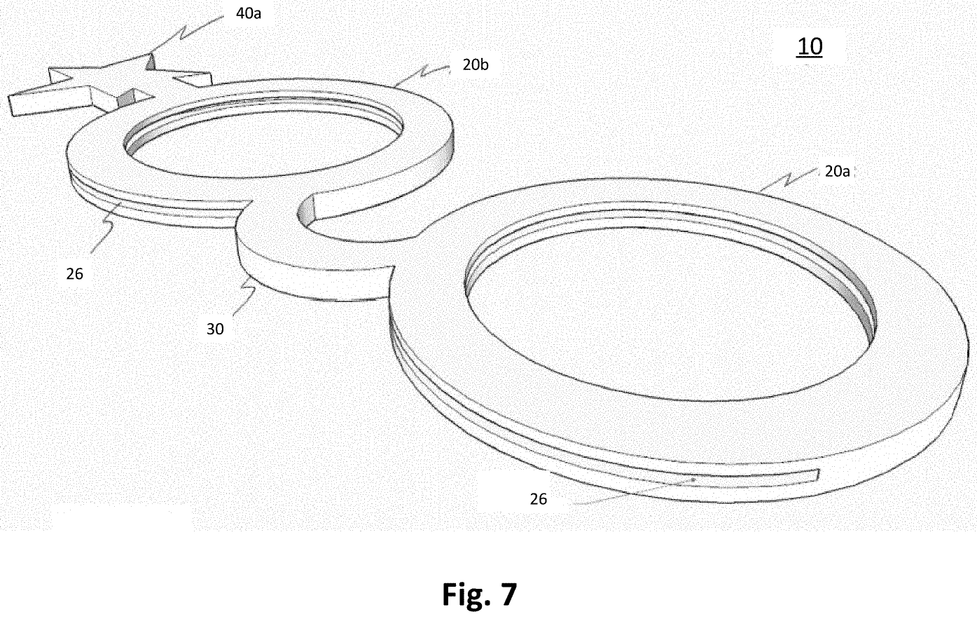

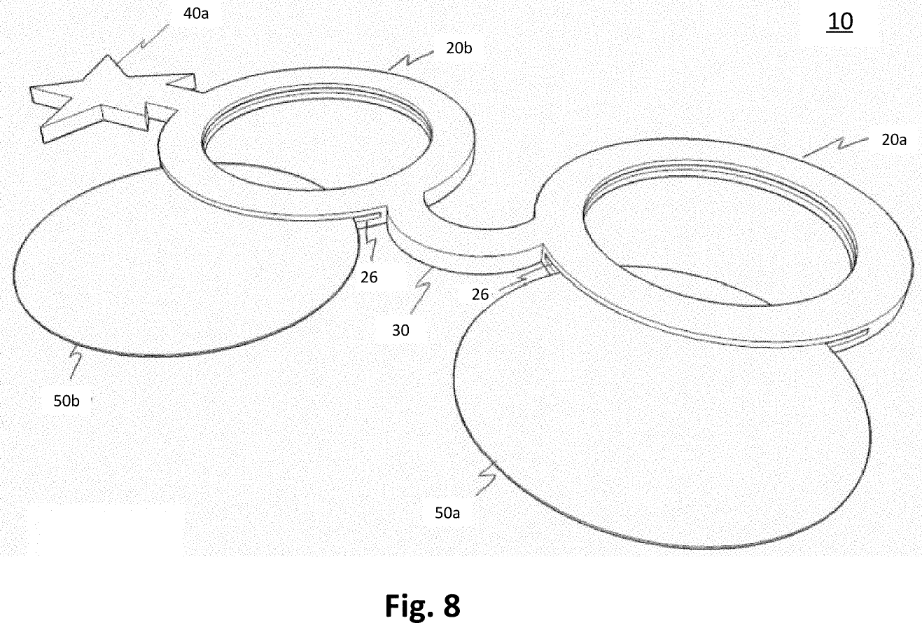

Another illustrative configuration of a frame 10 is shown in FIG. 7. In FIG. 7, the outer circumference of each of the sockets 20a, 20b includes a slit 26. Each slit 26 provides an opening between the front side and back side of a corresponding socket (20a or 20b) to allow a corresponding optical component (50a or 50b) to be inserted into the top side of each socket as illustrated in FIG. 8.

In other implementations it is possible to attach the optical component to the front side or rear side of the socket. Attachment can be achieved by using an adhesive or several fasteners such as a series of staples provided around the side of the socket to mount the optical component thereon.

It will be appreciated that each pair of sockets 20a, 20b can be circular or ring-like in shape, elliptical, oval or take on any other shape that is associated with a pair of eye glasses.

Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims.

1. A filter test kit for low vision comprising:
a plurality of frames, each frame comprising:
two sockets;
a pair of optical lens tint components disposed in the sockets;
a bridge connecting the sockets; and
a tab protruding from the frame to allow a user to hold the frame, wherein the tab includes a tactile frame identifier,
wherein the pair of optical lens tint components of each frame is different from the pair of optical lens tint components of each other frame, and
wherein the tactile frame identifier for the tab of each frame is different from the tactile frame identifier for the tab of each other frame.

2. The filter test kit according to claim 1, wherein each tactile frame identifier corresponds to a different shape.

3. The filter test kit according to claim 1, wherein the tactile frame identifier for each frame identifies the pair of optical lens tint components.

4. The filter test kit according to claim 1, further comprising:
an eye chart; and
a distance indicator having a length that corresponds to a distance at which a user should be from the eye chart to evaluate the pair of optical lens tint components.

5. The filter test kit according to claim 4, wherein the eye chart includes a contrast preference chart.

6. The filter test kit according to claim 1, wherein the tab protrudes from and extends away from a side of one of the sockets along an imaginary line that intersects the center of the sockets.

7. The filter test kit according to claim 1, wherein the sockets each comprise:
a base; and
a ring insert that press fits into the base of a socket, wherein the ring insert is configured to hold within the frame an optical lens tint component placed on the base when the ring insert is press fit to the base.

8. The filter test kit according to claim 1, wherein the sockets each comprise:
a slit on an outer circumference portion of a socket, the slit configured to allow an optical lens tint component to be inserted into a top side of the socket.

9. A filter test kit for low vision comprising:
a plurality of frames, each frame comprising:
two sockets;
a bridge connecting the sockets; and
a tab protruding from the frame to allow a user to hold the frame, wherein the tab includes a tactile frame identifier; and
a plurality of pairs of optical lens tint components, each pair of optical lens tint components configured to be disposed in the sockets of a different one of the frames,
wherein the pair of optical lens tint components for each frame is different from the pair of optical lens tint components for each other frame, and
wherein the tactile frame identifier for the tab of each frame is different from the tactile frame identifier for the tab of each other frame.

10. The filter test kit according to claim 9, wherein each tactile frame identifier corresponds to a different shape.

11. The filter test kit according to claim 9, wherein the tactile frame identifier for each frame corresponds to a different one of the pair of optical lens tint components.

12. The filter test kit according to claim 9, further comprising:
an eye chart; and
a distance indicator having a length that corresponds to a distance at which a user should be from the eye chart to evaluate each pair of optical lens tint components.

13. The filter test kit according to claim 12, wherein the eye chart includes a contrast preference chart.

14. The filter test kit according to claim 9, wherein the tab protrudes from and extends away from a side of one of the sockets along an imaginary line that intersects the center of the sockets.

15. The filter test kit according to claim 9, wherein the sockets each comprise:
a base; and
a ring insert that press fits into the base of a socket, wherein the ring insert is configured to hold within the frame an optical lens tint component placed on the base when the ring insert is press fit to the base.

16. The filter test kit according to claim 9, wherein the sockets each comprise:
a slit on an outer circumference portion of a socket, the slit configured to allow an optical lens tint component to be inserted into a top side of the socket.

17. A filter test frame for low vision comprising:
two sockets;
a pair of optical lens tint components disposed in the sockets;
a bridge connecting the sockets; and
a tab protruding from the filter test frame to allow a user to hold the filter test frame, wherein the tab includes a tactile frame identifier,
wherein the tactile frame identifier corresponds to a shape that identifies tint of the pair of optical lens tint components.

18. The filter test frame according to claim 17, wherein the tab protrudes from and extends away from a side of one of the sockets along an imaginary line that intersects the center of the sockets.

19. The filter test frame according to claim 17, wherein the sockets each comprise:
a base; and
a ring insert that press fits into the base of a socket, wherein the ring insert is configured to hold within the filter test pair frame an optical lens tint component placed on the base when the ring insert is press fit to the base.

20. The filter test frame according to claim 17, wherein the sockets each comprise:
a slit on an outer circumference portion of a socket, the slit configured to allow an optical lens tint component to be inserted into a top side of the socket.
